(19) United States
(12) Patent Application Publication
(10) Pub. No.: US 2026/0331282 A1
(43) Pub. Date: Oct. 22, 2026

(54) METHOD AND SYSTEM FOR CLASSIFICATION OF AN OCCURRENCE OF A MEDICAL COMPLICATION

(71) Applicant: Charité - Universitätsmedizin Berlin Körperschaft des Öffentlichen Rechts, Berlin (DE)

(72) Inventors: Stefan Verlohren, Hamburg (DE); Oliver Reiger, Berlin (DE); Leon Schmidt, Augsburg (DE)

(21) Appl. No.: 19/163,521

(22) PCT Filed: Dec. 23, 2024
(86) PCT No.: PCT/EP2024/088352

§ 371 Date: Sep. 9, 2025

(30) Foreign Application Priority Data
Jan. 9, 2024 (JP) 10 2024 100 425.8

(51) Int. Cl. G16H 50/20; G16H 10/65

(52) U.S. Cl. CPC G16H 50/20; G16H 10/65

(57) ABSTRACT

The invention relates to a computer-implemented method for classification of an occurrence of a complication caused by preeclampsia within a predetermined time period, comprising applying (S3) a second machine learning algorithm (A2) to the second dataset (DS2) of medical parameters of the patient to classify the occurrence of the complication caused by preeclampsia within the predetermined time period, and outputting (S4) a class representing the occurrence of the complication caused by preeclampsia within the predetermined time period. The invention further relates to a system (1) for classification of an occurrence of a complication caused by preeclampsia within a predetermined time period.

The present invention relates to a computer-implemented method for classification of an occurrence of a complication caused by preeclampsia within a predetermined time period.

The present invention further relates to a system for classification of an occurrence of a complication caused by preeclampsia within a predetermined time period.

Moreover, the invention relates to a computer program with program code to execute the inventive method, as well as a computer-readable medium with program code of a computer program to execute at least parts of the inventive method when the computer program is executed on a computer.

### STATE OF THE ART

Preeclampsia is a multisystem disease in pregnancy. With an incidence of 3-5%, it is one of the leading causes of maternal and fetal morbidity and mortality. Preeclampsia is defined as an increase in blood pressure during pregnancy in combination with the new onset of maternal organ symptoms such as proteinuria, liver dysfunction, neurological symptoms, and others. The prediction of preeclampsia is currently conducted according to valid national and international guidelines through regular measurement of maternal blood pressure and determination of proteinuria.

However, it is a clinical dilemma that these diagnostic criteria are too imprecise to predict severe courses or complications of preeclampsia. Due to the discrepancy between nonspecific symptoms and life-threatening complications, accurate prediction is crucial to prevent these.

An improvement in the prediction of preeclampsia was achieved with the establishment of angiogenic and antiangiogenic factors. It has been shown that the circulating serum concentration of the antiangiogenic factor soluble fms-like tyrosine kinase 1 (sFlt-1) as well as that of the angiogenic factor placental growth factor (PlGF) in the serum of women is altered who will develop preeclampsia.

In Zeisler H, Llurba E, Chantraine F, Vatish M, Staff AC, Sennström M, et al., “Predictive Value of the sFlt-1: PIGF Ratio in Women with Suspected Preeclampsia,” N Engl J Med. 2016; 374(1): 13-22, it was demonstrated that using the sFlt-1/PIGF ratio, a disease exclusion can be achieved for women presenting with clinical symptoms.

Inclusion was limited due to a moderate positive predictive value.

Thus, high prediction accuracy is desirable to reduce morbidity and mortality as well as to avoid unnecessary hospitalizations, thereby alleviating the burden on the healthcare system.

It is therefore the object of the invention to provide an improved method that enables more accurate determination of an occurrence of a complication caused by preeclampsia during pregnancy.

### DISCLOSURE OF THE INVENTION

The object is achieved according to the invention by a computer-implemented method for classification of an occurrence of a complication caused by preeclampsia within a predetermined time period with the features of claim 1.

The object is further achieved according to the invention by a system for classification of an occurrence of a complication caused by preeclampsia within a predetermined time period with the features of claim 11. The object is also achieved according to the invention by a computer program with program code to execute the inventive method, with the features of claim 14.

Additionally, the object is achieved by a computer-readable medium with program code of a computer program to execute at least parts of the inventive method when the computer program is executed on a computer, with the features of claim 15.

The invention relates to a computer-implemented method for classification of an occurrence of a complication caused by preeclampsia within a predetermined time period.

The method comprises providing a pre-recorded first dataset of medical parameters of a patient.

Furthermore, the method comprises at least partially transforming the first dataset of medical parameters of the patient using a first algorithm, in particular a multivariate data analysis method, into a reduced second dataset of medical parameters of the patient.

The method further comprises applying a second machine learning algorithm to the second dataset of medical parameters of the patient to classify the occurrence of the complication caused by preeclampsia within the predetermined time period, and outputting a class representing the occurrence of the complication caused by preeclampsia within the predetermined time period.

The invention further relates to a system for classification of an occurrence of a complication caused by preeclampsia within a predetermined time period. The system comprises at least one input device for providing a pre-recorded first dataset of medical parameters of a patient.

Moreover, the system comprises a first computing device configured to transform the first dataset of medical parameters of the patient using a first algorithm, in particular a multivariate data analysis method, into a reduced second dataset of medical parameters of the patient at least partially.

The system further comprises a second computing device configured to apply a second machine learning algorithm to the second dataset of medical parameters of the patient to classify the occurrence of the complication caused by preeclampsia within the predetermined time period, and an output device configured to output a class representing the occurrence of the complication caused by preeclampsia within a predetermined time period.

The invention also relates to a computer program with program code to execute a method for classification of an occurrence of a complication caused by preeclampsia within a predetermined time period when the computer program is executed on a computer, as well as a computer-readable medium with program code of a computer program to execute at least parts of the inventive method when the computer program is executed on a computer.

Machine learning algorithms are based on the use of statistical methods to train a data processing system to perform a specific task without being explicitly programmed for it initially. The goal of machine learning is to construct algorithms that can learn from data and make predictions. These algorithms create mathematical models that can, for example, classify data.

The first algorithm, in particular the multivariate data analysis method, performs the transformation of the first dataset of medical parameters of the patient into the reduced second dataset of medical parameters of the patient by feature selection and feature processing.

Relevant features are selected from the dataset and transformed into a format that can be used by the second machine learning algorithm. This can comprise feature extraction, feature scaling, and feature normalization.

An idea of the present invention is to use a machine learning algorithm in the field of risk prediction for pregnant women with high risk for preeclampsia. In case of a prediction of a possible complication, it may be recommended, for example, to terminate the pregnancy. This can eliminate the risk of the complication. The prediction can be performed throughout the course of pregnancy, preferably after the 24th week of pregnancy, or whenever a high-risk patient presents herself.

The machine learning algorithm is particularly applied to a reduced dataset of medical parameters of the patient. Overall, the machine learning model functions by learning from data, identifying patterns and relationships between variables, and making predictions or decisions based on them. The accuracy of the model depends on the quality and quantity of the data, the algorithm used, and the hyperparameter settings.

Feature selection and engineering are critical steps in creating machine learning models, as they aim to select the most important features from the dataset and convert them into a format that the machine learning algorithm can use.

During feature selection, a subset of relevant features is selected to be used in the model. The goal of feature selection is to reduce the dimensionality of the data and remove all redundant or irrelevant features that could negatively impact the model's performance.

The classification of the occurrence of the complication caused by preeclampsia within the predetermined time period can, for example, be provided by a binary classification distinguishing only between a prediction of the occurrence of a complication and a non-occurrence of a complication.

Alternatively, the classification can, for example, output a percentage probability of the occurrence of a complication, expressed as a numerical value between 0 and 1.

Additionally, a platform is created that integrates a physician-side decision support tool with a patient-side home monitoring application.

Further embodiments of the present invention are the subject of the additional claims and the following description with reference to the figures.

According to a preferred embodiment of the invention, it is provided that the first dataset comprises a first set of medical parameters of the patient recorded through a user interface of a medical practitioner, comprising protein levels in urine, systolic blood pressure, diastolic blood pressure, an sFlt-1 value, a PIGF value, a classification of thrombocytopenia, and a classification of elevated liver enzymes.

Part of the features comprised by the first dataset can thus advantageously be provided by the medical practitioner through the user interface.

According to another preferred embodiment of the invention, it is provided that the first dataset comprises a second set of medical parameters of the patient recorded through a patient app and/or the user interface of the medical practitioner, comprising the patient's age, weight before the current pregnancy, height, a classification of hypertension before the current pregnancy, a classification of new onset hypertension during the current pregnancy, and gestational age, particularly in days.

Another part of the features comprised by the first dataset can thus advantageously be provided by the patient through the patient app.

According to another preferred embodiment of the invention, it is provided that the second machine learning algorithm performs a further classification of the occurrence of the complication caused by preeclampsia within the predetermined time period based on the re-provision of medical parameters of the patient recorded by the patient app.

Thus, the risk of the occurrence of the complication caused by preeclampsia can advantageously be continuously re-determined based on updated medical parameters provided by the patient app.

According to another preferred embodiment of the invention, it is provided that the transformed second dataset comprises the patient's age, body mass index before pregnancy, multiples of the median of sFlt-1, multiples of the median of PIGF, multiples of the median of the sFlt-1/PIGF ratio, a deviation from the mean systolic blood pressure, and a deviation from the mean diastolic blood pressure.

The transformed second dataset thus advantageously contains relevant features and a format that can be used by the second machine learning algorithm.

According to another preferred embodiment of the invention, it is provided that the second machine learning algorithm and/or a third machine learning algorithm applied to the second dataset of medical parameters of the patient outputs a classification of a delivery caused by preeclampsia within a predetermined time period. This prediction advantageously provides an improved basis for decision-making regarding any necessary medical interventions.

According to another preferred embodiment of the invention, it is provided that the second machine learning algorithm and/or the third machine learning algorithm applied to the second dataset of medical parameters of the patient outputs the classification of a delivery caused by preeclampsia within the next 14 days for patients before the 34th week of pregnancy and the classification of a delivery caused by preeclampsia within the next 7 days for patients after the 34th week of pregnancy.

Based on this determination, the physician can make a better decision about whether the patient is at high risk and needs to be treated in a hospital, whether the patient should be delivered immediately or very soon, or whether, despite symptoms, the patient can be sent home with a higher level of certainty than previously possible.

According to another preferred embodiment of the invention, it is provided that the second machine learning algorithm and/or a fourth machine learning algorithm applied to the second dataset of medical parameters of the patient outputs a classification of an occurrence of preeclampsia.

The prediction of the occurrence of preeclampsia advantageously provides the physician with additional information required for treatment. For example, the occurrence of preeclampsia and simultaneously the non-occurrence of a complication caused by preeclampsia can be predicted. This enables improved decision-making for a possible treatment of the patient.

According to another preferred embodiment of the invention, a computer-implemented method is provided, further comprising the provision of the classification generated by the second machine learning algorithm, the third machine learning algorithm, and/or the fourth machine learning algorithm.

The provided classification result can then, for example, be transmitted to the user interface of the medical practitioner and/or the patient app.

According to another preferred embodiment of the invention, it is provided that the classification of the occurrence of the complication caused by preeclampsia within the predetermined time period comprises a first class, which represents a prediction of the occurrence of the complication caused by preeclampsia within the predetermined time period, and a second class of the classification represents a prediction of the non-occurrence of the complication caused by preeclampsia within the predetermined time period.

The binary classification thus allows a concrete statement regarding the occurrence or non-occurrence of the complication caused by preeclampsia within the predetermined time period.

According to another preferred embodiment of the invention, it is provided that the at least one input device comprises a user interface of a medical practitioner configured to provide a first set of medical parameters of the patient recorded by the medical practitioner, comprising protein levels in urine, systolic blood pressure, diastolic blood pressure, an sFlt-1 value, a PIGF value, a classification of thrombocytopenia, and a classification of elevated liver enzymes.

Part of the features comprised by the first dataset can thus advantageously be provided by the medical practitioner through the user interface.

According to another preferred embodiment of the invention, it is provided that the at least one input device comprises a patient app configured to provide a second set of medical parameters recorded by the patient, comprising the patient's age, weight before the current pregnancy, height, a classification of hypertension before the current pregnancy, a classification of new onset hypertension during the current pregnancy, and gestational age, particularly in days.

Another part of the features comprised by the first dataset can thus advantageously be provided by the patient through the patient app.

The features described herein of the computer-implemented method for classification of an occurrence of a complication caused by preeclampsia within a predetermined time period are also applicable to the system for classification of an occurrence of a complication caused by preeclampsia within a predetermined time period and vice versa.

### BRIEF DESCRIPTION OF THE DRAWINGS

For a better understanding of the present invention and its advantages, reference is now made to the following description in conjunction with the accompanying drawings.

The invention will now be explained in more detail by way of exemplary embodiments indicated in the schematic illustrations of the drawings.

In the drawings:

FIG. 1 shows a flowchart of a computer-implemented method for classification of an occurrence of a complication caused by preeclampsia within a predetermined time period; and

FIG. 2 shows a schematic representation of a system for classification of an occurrence of a complication caused by preeclampsia within a predetermined time period.

Unless otherwise indicated, identical reference numerals designate identical elements in the drawings.

### DETAILED DESCRIPTION OF THE EMBODIMENTS

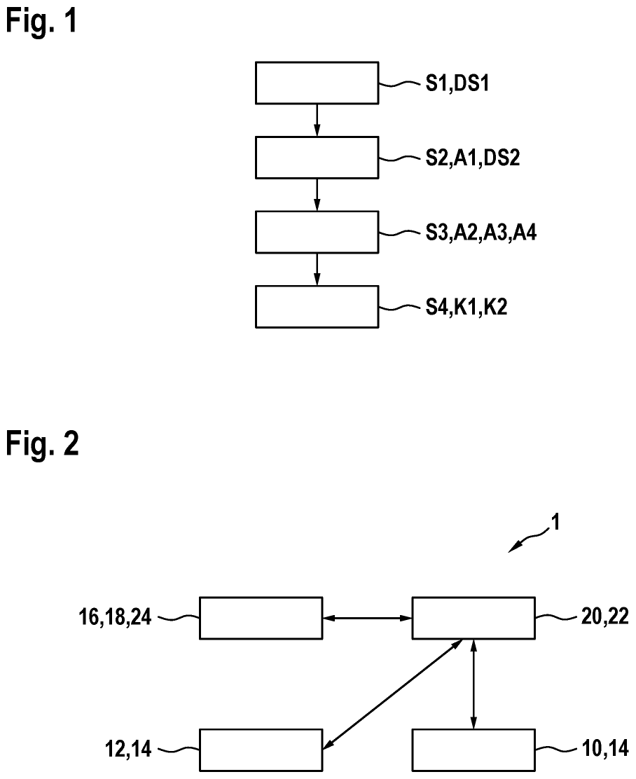

The computer-implemented method for classification of an occurrence of a complication caused by preeclampsia within a predetermined time period shown in FIG. 1 comprises providing S1 a pre-recorded first dataset DS1 of medical parameters of a patient.

Furthermore, the method comprises at least partially transforming S2 the first dataset DS1 of medical parameters of the patient using a first algorithm Al, in particular a multivariate data analysis method, into a reduced second dataset DS2 of medical parameters of the patient.

The method further comprises applying S3 a second machine learning algorithm A2 to the second dataset DS2 of medical parameters of the patient to classify the occurrence of the complication caused by preeclampsia within the predetermined time period.

Moreover, the method comprises outputting S4 a class K1, K2 representing the occurrence of the complication caused by preeclampsia within a predetermined time period. The first dataset DS1 comprises a first set of medical parameters of the patient recorded via a user interface 10 of a medical practitioner, comprising protein levels in urine, systolic blood pressure, diastolic blood pressure, an sFlt-1 value, a PIGF value, a classification of thrombocytopenia, and a classification of elevated liver enzymes.

The first dataset DS1 further comprises a second set of medical parameters of the patient recorded via a patient app 12 and/or the user interface 10 of the medical practitioner, comprising the patient's age, weight before the current pregnancy, height, a classification of hypertension before the current pregnancy, a classification of new onset hypertension during the current pregnancy, and gestational age, in particular in days.

The second machine learning algorithm A2 performs a further classification of the occurrence of the complication caused by preeclampsia within the predetermined time period based on the re-provision of medical parameters of the patient recorded via the patient app 12.

The transformed second dataset DS2 comprises the patient's age, body mass index before pregnancy, multiples of the median of sFlt-1, multiples of the median of PIGF, multiples of the median of the sFlt-1/PIGF ratio, a deviation from the mean systolic blood pressure, and a deviation from the mean diastolic blood pressure.

The second machine learning algorithm A2 and/or a third machine learning algorithm A3 applied to the second dataset DS2 of medical parameters of the patient further outputs a classification of a delivery caused by preeclampsia within a predetermined time period.

Furthermore, the second machine learning algorithm A2 and/or the third machine learning algorithm A3 applied to the second dataset DS2 of medical parameters of the patient outputs the classification of a delivery caused by preeclampsia within the next 14 days for patients before the 34th week of pregnancy and the classification of a delivery caused by preeclampsia within the next 7 days for patients after the 34th week of pregnancy.

The second machine learning algorithm A2 and/or a fourth machine learning algorithm A4 applied to the second dataset DS2 of medical parameters of the patient further outputs a classification of an occurrence of preeclampsia.

Furthermore, the method comprises providing the classification generated by the second machine learning algorithm A2, the third machine learning algorithm A3, and/or the fourth machine learning algorithm A4.

The classification of the occurrence of the complication caused by preeclampsia within the predetermined time period comprises a first class Kl, which represents a prediction of the occurrence of the complication caused by preeclampsia within the predetermined time period.

Furthermore, the classification comprises a second class K2, which represents a prediction of the non-occurrence of the complication caused by preeclampsia within the predetermined time period.

Alternatively, the classification may, for example, output a percentage probability of the occurrence of a complication, expressed as a numerical value between 0 and 1.

FIG. 2 shows a schematic representation of a system 1 for classification of an occurrence of a complication caused by preeclampsia within a predetermined time period.

The system 1 comprises at least one input device 14 for providing a pre-recorded first dataset DS1 of medical parameters of a patient.

Furthermore, the system 1 comprises a first computing device 16, which is configured to at least partially transform the first dataset of medical parameters of the patient using a first algorithm Al, in particular multivariate data analysis method, into a reduced second dataset DS2 of medical parameters of the patient.

The system 1 further comprises a second computing device 18, which is configured to apply a second machine learning algorithm A2 to the second dataset DS2 of medical parameters of the patient to classify the occurrence of the complication caused by preeclampsia within the predetermined time period.

In addition, the system 1 comprises an output device 20, which is configured to output a class representing the occurrence of the complication caused by preeclampsia within a predetermined time period.

The at least one input device 14 comprises a user interface 10 of a medical practitioner, which is configured to provide a first set of medical parameters of the patient recorded by the medical practitioner, comprising protein levels in urine, systolic blood pressure, diastolic blood pressure, an sFlt-1 value, a PIGF value, a classification of thrombocytopenia, and a classification of elevated liver enzymes.

Furthermore, the at least one input device 14 comprises a patient app 12, which is configured to provide a second set of medical parameters recorded by the patient, comprising the patient's age, weight before the current pregnancy, height, a classification of hypertension before the current pregnancy, a classification of new onset hypertension during the current pregnancy, and gestational age, particularly in days.

The user interface 10 of the medical practitioner and the patient app 12 are configured to communicate with a backend app 22. The backend app 22 is in turn connected to a model-serving app 24, wherein the provided medical parameters are transmitted from the backend app 22 to the model-serving app 24.

The classification generated by the corresponding machine learning model is then transmitted from the model-serving app 24 back to the backend app 22, which the user interface 10 of the medical practitioner and/or the patient app 12 can access.

The patient app 12 and/or the user interface 10 of the medical practitioner can be provided, for example, as a desktop app and/or smartphone app.

Although specific embodiments have been illustrated and described herein, it is understood by those skilled in the art that numerous alternative and/or equivalent implementations exist. It should be noted that the exemplary embodiment or exemplary embodiments are merely examples and are not intended to limit the scope, applicability, or configuration in any way.

Rather, the aforementioned summary and detailed description provide those skilled in the art with a convenient guide for implementing at least one exemplary embodiment, with the understanding that various changes in function and arrangement of elements may be made without departing from the scope of the appended claims and their legal equivalents.

In general, this application is intended to cover modifications, adjustments, or variations of the embodiments presented herein. For example, the order of the method steps may be altered. The method may also be performed at least partially sequentially or in parallel.

### LIST OF REFERENCE SIGNS

1 System
10 User interface of a medical practitioner
12 Patient app
14 Input device
16 First computing device
18 Second computing device
20 Output device
22 Backend app
24 Model-serving app
A1 First algorithm
A2 Second machine learning algorithm
A3 Third machine learning algorithm
A4 Fourth machine learning algorithm
DS1 First dataset
DS2 Second dataset
K1 First class
K2 Second class
S1-S4 Method steps

1-15. (canceled)

16. Computer-implemented method for classification of an occurrence of a complication caused by preeclampsia within a predetermined time period, comprising:
providing (S1) a pre-recorded first dataset (DS1) of medical parameters of a patient;
at least partially transforming (S2) the first dataset (DS1) of medical parameters of the patient using a first algorithm (A1), in particular a multivariate data analysis method, into a reduced second dataset (DS2) of medical parameters of the patient;
applying (S3) a second machine learning algorithm (A2) to the second dataset (DS2) of medical parameters of the patient to classify the occurrence of the complication caused by preeclampsia within the predetermined time period; and
outputting (S4) a class (K1, K2) representing the occurrence of the complication caused by preeclampsia within the predetermined time period.

17. Computer-implemented method according to claim 16, wherein the first dataset (DS1) comprises a first set of medical parameters of the patient recorded by a user interface (10) of a medical practitioner, comprising protein levels in urine, systolic blood pressure, diastolic blood pressure, an sFlt-1 value, a PIGF value, a classification of thrombocytopenia, and a classification of elevated liver enzymes.

18. Computer-implemented method according to claim 17, wherein the first dataset (DS1) comprises a second set of medical parameters of the patient recorded by a patient app (12) and/or the user interface (10) of the medical practitioner, comprising the patient's age, weight before the current pregnancy, height, a classification of hypertension before the current pregnancy, a classification of new onset hypertension during the current pregnancy, and gestational age, in particular in days.

19. Computer-implemented method according to claim 18, wherein the second machine learning algorithm (A2) performs a further classification of the occurrence of the complication caused by preeclampsia within the predetermined time period based on the re-provision of medical parameters of the patient recorded by the patient app (12).

20. Computer-implemented method according to claim 16, wherein the transformed second dataset (DS2) comprises the patient's age, body mass index before pregnancy, multiples of the median of sFlt-1, multiples of the median of PIGF, multiples of the median of the ratio of sFlt-1 and PIGF, a deviation from the mean systolic blood pressure, and a deviation from the mean diastolic blood pressure.

21. Computer-implemented method according to claim 20, wherein the second machine learning algorithm (A2) and/or a third machine learning algorithm (A3) applied to the second dataset (DS2) of medical parameters of the patient outputs a classification of a delivery caused by preeclampsia within a predetermined time period.

22. Computer-implemented method according to claim 21, wherein the second machine learning algorithm (A2) and/or the third machine learning algorithm (A3) applied to the second dataset (DS2) of medical parameters of the patient outputs the classification of a delivery caused by preeclampsia within the next 14 days for patients before the 34th week of pregnancy and the classification of a delivery caused by preeclampsia within the next 7 days for patients after the 34th week of pregnancy.

23. Computer-implemented method according to claim 20, wherein the second machine learning algorithm (A2) and/or a fourth machine learning algorithm (A4) applied to the second dataset (DS2) of medical parameters of the patient outputs a classification of an occurrence of preeclampsia.

24. Computer-implemented method according to claim 23, further comprising providing the classification generated by the second machine learning algorithm (A2), the third machine learning algorithm (A3), and/or the fourth machine learning algorithm (A4).

25. Computer-implemented method according to claim 16, wherein the classification of the occurrence of the complication caused by preeclampsia within the predetermined time period comprises a first class (K1) representing a prediction of the occurrence of the complication caused by preeclampsia within the predetermined time period, and wherein a second class (K2) of the classification represents a prediction of the non-occurrence of the complication caused by preeclampsia within the predetermined time period.

26. System (1) for classification of an occurrence of a complication caused by preeclampsia within a predetermined time period, comprising:
at least one input device (14) for providing a pre-recorded first dataset (DS1) of medical parameters of a patient;
a first computing device (16) configured to transform the first dataset of medical parameters of the patient using a first algorithm (A1), in particular a multivariate data analysis method, into a reduced second dataset (DS2) of medical parameters of the patient at least partially;
a second computing device (18) configured to apply a second machine learning algorithm (A2) to the second dataset (DS2) of medical parameters of the patient to classify the occurrence of the complication caused by preeclampsia within the predetermined time period; and
an output device (20) configured to output a class (K1, K2) representing the occurrence of the complication caused by preeclampsia within the predetermined time period.

27. System according to claim 26, wherein the at least one input device (14) comprises a user interface (10) of a medical practitioner configured to provide a first set of medical parameters of the patient recorded by the medical practitioner, comprising protein levels in urine, systolic blood pressure, diastolic blood pressure, an sFlt-1 value, a PIGF value, a classification of thrombocytopenia, and a classification of elevated liver enzymes.

28. System according to claim 26, wherein the at least one input device (14) comprises a patient app (12) configured to provide a second set of medical parameters recorded by the patient, comprising the patient's age, weight before the current pregnancy, height, a classification of hypertension before the current pregnancy, a classification of new onset hypertension during the current pregnancy, and gestational age, in particular in days.

29. Computer program with program code to execute at least parts of a method according to claim 16, when the computer program is executed on a computer.

30. Computer-readable medium with program code of a computer program to execute at least parts of a method according to claim 16, when the computer program is executed on a computer.
